(19) United States
(12) Patent Application Publication
(10) Pub. No.: US 2020/0188655 A1
(43) Pub. Date: Jun. 18, 2020

(54) NON-FIBROTIC BIOCOMPATIBLE ELECTRODE AND RELATED METHODS

(71) Applicant: Arizona Board of Regents on behalf of Arizona State University, Scottstale, AZ (US)

(72) Inventors: Mehdi Nikkhah, Scottsdale, AZ (US); Mark Allen, Mesa, AZ (US)

(21) Appl. No.: 16/611,149

(22) PCT Filed: May 8, 2018
(86) PCT No.: PCT/US2018/031608

§ 371 Date: Nov. 5, 2019

Related U.S. Application Data
(60) Provisional application No. 62/503,710, filed on May 9, 2017.

(51) Int. Cl. A61N 1/05; A61L 27/02; A61L 27/44; A61L 27/52; A61L 27/34; C08L 71/02

(52) U.S. Cl. CPC A61N 1/056; A61N 1/0551; A61L 27/02; A61L 27/44; A61L 2420/04; A61L 27/34; C08L 71/02; A61L 2420/08; A61L 27/52

(57) ABSTRACT

Electrodes comprising an electrode coated with a coating, the coating comprising a non-fibrotic material, wherein the non-fibrotic material comprises electrically conductive particles dispersed therein, are provided. The non-fibrotic material may comprise hydrogel lacking cell adhesion moieties. The hydrogel may comprise poly(ethylene) glycol. The electrically conductive particles may comprise gold. Such electrodes may provide electrical stimulation to tissues, while eliminating or reducing fibrosis of tissue coming into contact with the electrodes. Such electrodes may accomplish these ends without the use of drugs. Such electrodes may be useful in applications in which electrical stimulation of tissues is used, such as in cardiac pacemakers, neural stimulators, and muscle stimulators. Methods of making and of evaluating such electrodes are provided.

This application claims priority to U.S. provisional application No. 62/503,710, which was filed May 9, 2017 and is entitled “Non-Fibrotic Biocompatible Electrode And Related Methods.” The 62/503,710 application is incorporated herein in its entirety. This application is a National Stage Entry of application PCT/US2018/031608, filed May 8, 2018. The PCT/US2018/031608 application is incorporated herein in its entirety. In the PCT/US2018/031608 application, a Substitute Sequence Listing having the file name “M17-133L-WO1_Seq_List_ST25_6-14-18.txt” and the file creation date “Nov. 5, 2019” was submitted on Jun. 14, 2018. This Substitute Sequence Listing is incorporated herein in its entirety.

### TECHNICAL FIELD

This application relates generally to electrodes that have reduced tendency to induce fibrosis of surrounding tissue or are non-fibrotic.

### TECHNICAL BACKGROUND

Millions of patients around the world have some form of implantable pacemaker (PM) device to control cardiac arrhythmias, with more and more implantations every year. Globally the rate of PM implantation is increasing, with 2.9 million patients receiving a PM from 1993-2009. Depending on the study data, early complications are reported in 4-5% of the recipients while late complications occur 2.7% of the time. The release of fibroblasts, leukocytes, phagocytes, oxidants, and other foreign body activity cause the formation of a fibrotic capsule at the electrode-tissue interface and shorten the device's battery life, resulting in more frequent surgical procedures that can create additional patient complications including death. This general myofibrillar disarray can also lead to dissolution of the extracellular scaffold and cause necrosis in adjacent myocytes. Because of these factors, the maximum PT generally spikes at approximately three weeks post-implant and is two to three times greater than what is necessary to stimulate the heartbeat. The PT will then stabilize once it has compensated for the increased insulation caused by the formation of the fibrotic capsule, but at an output higher than needed or desired. Moreover, the damage to the tissue is permanent and can even lead to death in a number of patients. Mechanical injury from the implant may also initiate a biological response.

A host of complications can arise as a result of the implantation, such as lead or electrode dislodgement, pnuemothorax, lead or electrode perforation, or venous thrombosis. Frequent implantation complications include pocket infections or endocarditis, which can be especially problematic as biofilms can rapidly accumulate in the area. However, the most frequent implantation complication is the foreign body response that results in the formation of a fibrotic capsule around the electrode and/or lead. Even with modern electrodes and drug release enhancements, fibrotic capsules up to 100 μm tend to build up around the implanted device in just the first few weeks. This reaction to the device can oftentimes cause pain and discomfort to the patient and impede its performance. Due to the increase in voltage needed to overcome the development of the fibrotic capsule, a positive feedback loop can create even more fibrosis. In addition, the device battery is drained more quickly due to the increased current and requires more frequent replacement, which requires a surgical procedure. In most cases, permanent damage to the myocardium arises and, in some cases, even patient death can occur. As a result, there is still a need to develop new materials or methods in order to suppress fibrosis from PM electrodes and/or leads. Despite decades of materials research, a non-fibrotic, conductive, implantable cardiac pacemaker electrode and/or lead has yet to be developed. Although substantial progress has been made in the synthesis of biomaterial surface coatings, a coating material that is nonreactive to the extracellular matrix (ECM) has not yet been developed.

### SUMMARY

Electrodes and/or leads may be used in medical and other biological applications to conduct electricity to tissue, such as heart tissue, brain, nerves, or other neurological tissue, and/or muscle tissue. However, undesirable tissue may build up around the electrode and/or leads where it is in contact with the tissue intended to receive electrical stimulation. Electrodes and/or leads are described herein that may comprise a biocompatible coating that may prevent or reduce such buildup of undesirable tissue, while still conducting electricity to the tissue intended to receive electrical stimulation. The coated electrodes and/or leads may achieve such prevention or reduction of buildup of undesirable tissue without the use of drugs, or with a smaller or less frequent use of drugs, as compared to uncoated electrodes. The term “uncoated electrode” or “uncoated lead” may refer to herein to an electrode or lead that lacks a coating as described herein, i.e., a coating comprising a non-fibrotic material (as later defined herein) and/or an electrically conductive material (as later defined herein). Such coated electrodes and/or leads may be useful in medical or other biological applications in which electrical stimulation of tissues is used, such as in cardiac pacemakers, neural stimulators, and muscle stimulators. Methods of making and of evaluating the performance of such electrodes and/or leads are also provided.

Embodiments may include an electrode coated with a coating, the coating comprising non-fibrotic material, wherein the non-fibrotic material comprises electrically conductive particles dispersed therein. The coating may comprise two or more layers, and the two or more layers may differ in composition, thickness, or both. The dispersion may be random or ordered.

Embodiments may include an electrode, at least a portion of which is coated with a coating, the coating comprising (1) an inner layer comprising a non-fibrotic material, wherein the non-fibrotic material comprises electrically conductive particles dispersed therein, and (2) an outer layer comprising a non-fibrotic material, wherein the outer layer of non-fibrotic material does not have electrically conductive particles dispersed therein. The coating may comprise an innermost layer comprising a hydrogel lacking cell adhesion moieties. The coating may comprise additional layers, and the additional layers may differ in composition, organization, and/or thickness. Dispersions may be random or ordered.

Embodiments may include an electrode, at least a portion of which is coated with alternating layers of (1) a non-fibrotic material and (2) electrically conductive particles, which may be referred to herein as a “layer-by-layer” or “(LBL)” construction, as contrasted to a dispersion. Embodiments may include a lead that is coated partially or entirely with a coating comprising a non-fibrotic material. The coating may comprise additional layers, and the additional layers may differ in composition, organization, and/or thickness.

Coatings combining both LBL layers and dispersion layers by be utilized, in embodiments. In some embodiments, the electrically conductive particles may comprise gold. the electrically conductive particles may comprise nanowires, nanorods, and/or nanoparticles. The nanowires, nanorods, and/or nanoparticles may comprise gold. The non-fibrotic material may be a hydrogel lacking cell adhesion moieties. The hydrogel lacking cell adhesion moieties may comprise poly(ethylene) glycol. The hydrogel lacking cell adhesion moieties may comprise thiolated poly(ethylene) glycol. In some embodiments, the hydrogel lacking cell adhesion moieties may not comprise alginate.

The coating may be immobilized on the electrode via covalent binding. The coating may be immobilized on the electrode via non-covalent binding. The coating may be immobilized on the electrode via a peptide that binds both the (i) electrode and (ii) the hydrogel, the electrically conductive material, or both. The peptide that binds both the (i) electrode and (ii) the hydrogel, the electrically conductive material, or both may be a metal binding peptide, such as a titanium binding peptide.

In some embodiments, the electrode may be one that is capable of successfully delivering an electrical current to support contractility of cardiomyocytes, to stimulate neural tissue, and/or to stimulate muscle tissue. The electrode may comprise titanium, iridium, platinum, silicon, carbon, or a combination thereof. The electrode may be a TI-6AL-4V electrode or other electrode comprising titanium, or an Ir(IV)O₂ electrode or other electrode comprising iridium.

In some embodiments, the electrode tip or other portions of the electrode may be coated. The coating may cover all, most, or a portion of the electrode that comes into contact with tissue. In embodiments, all or portions of an electrode lead, such as portions that comes into contact with tissue, may also be coated.

Embodiments may include methods of evaluating the performance characteristics of a coated electrode and/or lead, wherein the performance characteristics can comprise the electrical conductivity of the coated electrode and/or lead, the resistance to fibrosis of the coated electrode and/or lead, the biological stability of the coated electrode and/or lead, the mechanical stability of the coated electrode and/or lead, or a combination thereof. The method may comprise the steps of: (a) (i) seeding cultured cells on one or more electrode and/or lead coated with a first coating or (ii) implanting one or more electrode and/or lead coated with a first coating in an animal model, such that the one or more electrode and/or lead is in contact with cells of the animal; (b) electrically stimulating the cells seeded on the electrode or contacting the electrode and/or lead in the animal model via the one or more electrode and/or lead coated with the first coating; and (c) observing one or more of the electrical conductivity, the resistance to fibrosis, the biological stability, and the mechanical stability of the one or more electrode and/or lead coated with the first coating. The cells may be observed for a period of days, weeks, or months, such as approximately 1-6 weeks, approximately 3-5 weeks, approximately 2, weeks, or approximately 4 weeks. The cells or animal model may be observed for a period of days, weeks, or months, such as approximately 8-12 weeks. The cells may be stimulated more than once. Methods may further comprise the steps of: (d) altering the one or more of the structure, number of layers, or composition of layers of coating to form a second coating; (e) repeating steps (a)-(c) with one or more electrode and/or lead coated with the second coating; and (f) comparing performance characteristics of the one or more electrode and/or lead coated with the first coating to the performance characteristics of the one or more electrode and/or lead coated with the second coating.

Embodiments may include methods of preparing an electrode coated with a coating comprising (1) a random dispersion of electrically conductive material in a hydrogel lacking cell adhesion moieties or (2) alternating layers of a hydrogel lacking cell adhesion moieties and electrically conductive particles comprising the steps of: (a) synthesizing of seeds of electrically conductive material; (b) synthesizing nanowires of electrically conductive material; (c)(i) dispersing the nanowires in the non-fibrotic material; and (ii) immobilizing the non-fibrotic material comprising the nanowires dispersed therein on the surface of the electrode in one or more area(s) where the electrode, once implanted into a tissue, will be in contact with the tissue; or (d)(i) immobilizing the non-fibrotic material or the nanowires on the surface of the electrode in one or more area(s) where the electrode, once implanted into a tissue, will be in contact with the tissue; and (ii) layering the nanowires on the immobilized non-fibrotic material or layering the non-fibrotic material on the immobilized nanowires, alternating two or more layers of nanowires and non-fibrotic material.

Embodiments may include methods of preparing a lead coated with a coating comprising a non-fibrotic material comprising the step of: immobilizing the non-fibrotic material on the surface of the lead in one or more area(s) where the lead, once implanted into a tissue, will be in contact with the tissue.

Various features, steps, processes, components, and subcomponents as may be employed in embodiments are provided herein. These features, steps, processes, components, subcomponents, partial steps, systems, devices, etc. may be adjusted, combined and modified in various fashions and various ways among and between the teachings and figures provided herein, as well as in other ways not specifically described herein but consistent with the teachings and discussion of this disclosure.

### BRIEF DESCRIPTION OF THE DRAWINGS

The accompanying drawings illustrate embodiments of the invention and, together with a general description of the invention given above, and the detailed description given below, serve to explain principles of the invention.

FIG. 1 illustrates a pacemaker assembly with electrode leads and electrodes implanted in a heart, in accordance with some embodiments.

FIG. 2 illustrates a cross section of a coated electrode in accordance with some embodiments.

FIG. 3 illustrates a cross section of a coated electrode, wherein the coating comprises layers having different compositions, in accordance with some embodiments.

FIG. 4 illustrates a cross section of a second coated electrode, wherein the coating comprises layers having different compositions, in accordance with some embodiments.

FIG. 5a illustrates development of a GNW-PEG coating with a layer-by-layer construction using poly(ethylene) glycol (PEG) layered with gold nanowires (GNW), in accordance with some embodiments.

FIG. 5b illustrates development of a GNW-PEG coating with a random dispersion of GNW in PEG matrix, in accordance with some embodiments.

FIG. 5c illustrates development of a titanium electrode coated with a layer-by-layer construction of PEG layered with gold nanowires (GNW), in accordance with some embodiments. The electrode and coating are shown in cross-section.

FIG. 5d illustrates development of a titanium electrode coated with a coating immobilized to the titanium by a surface chemistry, in accordance with some embodiments. The electrode, surface chemistry, and coating are shown in cross-section.

### DETAILED DESCRIPTION

Combating implantation fibrosis requires a new approach to biomaterials design. Successful development of biomaterials for a pacemaker electrode and/or lead is strongly dependent on its electrical properties and the response to the material-tissue interface.

Embodiments may include devices, systems, processes, and articles of manufacture relating to conductive, biocompatible electrodes and/or leads. These electrodes and/or leads may eliminate or reduce fibrotic response of tissue upon implantation or contact with tissue. These electrodes and/or leads may be coated. This coating might be a complete or partial coating of a lead or electrode and may vary depending on the application or specific location of placement of the lead or electrode, such as cardiac applications and neural or muscular applications. The coating may eliminate or reduce fibrotic response of tissue upon implantation or contact with tissue but may not reduce the conductive ability of the electrode and/or lead or may reduce it somewhat but still retain enough conductivity to perform the intended function of the electrode and/or lead. In embodiments, the intended function of the electrode may be successfully delivering an electrical current to support contractility of cardiomyocytes, successfully delivering a therapeutic current to neural cells, and/or successfully delivering a therapeutic current to muscle cells. In embodiments, the intended function of the lead may be successfully conducting an electrical current to an electrode implanted in cardiomyocytes, successfully conducting an electrical current to an electrode implanted in neural cells, and/or successfully conducting an electrical current to an electrode implanted in muscle cells, to support the successfully delivery of a current to such cells by an electrode. Successful delivery may be the delivery of the voltage, amperage, and/or frequency of electricity intended to be delivered; or the delivery of a therapeutic voltage, amperage, and/or frequency of electricity. A voltage, amperage, and/or frequency of electricity may be considered to be therapeutic if it is a standard or intended voltage, amperage, and/or frequency of electricity, or one that commonly results in a therapeutic response, even if a particular patient fails to realize a therapeutic response.

A conductive and biocompatible electrode, such as titanium-based electrode, and/or lead when coated with a novel coating comprising a non-fibrotic material (as later defined herein) and/or an electrically conductive material (as later defined herein), may lead to two major beneficial aspects; a) the electrically conductive material may provide excellent conductivity from the electrode, such as a Ti-6Al-4V electrode, and b) the non-fibrotic material may create a neutral, inert surface that may resist/reduce or eliminate/prevent a fibrotic response in the surrounding tissue. In embodiments, reduction of a fibrotic response may include: a reduction in numbers of fibrotic cells and/or proteins adhered or adsorbed to the electrode and/or lead; a reduction in thickness of any fibrotic capsule formed; a reduction in the length or other dimension of any fibrotic capsule formed; fewer fibroblasts adhered to the electrode and/or lead; less extracellular matrix deposition on the electrode and/or lead; and/or less activation of fibroblast cells to myo-fibroblast cells; each as compared to the fibrosis commonly and regularly observed with uncoated electrodes and/or leads. For example, fibrotic capsules of up to several hundreds of microns in thickness are commonly observed with uncoated electrodes and/or leads. Other examples of benefits of coated electrodes and/or leads may include elimination or reduction of occurrence, frequency, or severity of pocket infections, endocarditis, and/or damage to cardiac tissue. Other examples of benefits of coated electrodes and/or leads may include elimination or reduction of occurrence, frequency, or severity of electrode and/or lead dislodgement, pnuemothorax, electrode and/or lead perforation, venous thrombosis, and/or immune response. The biomaterial coating may prevent, lessen, or ameliorate adverse and fibrotic reactions to the electrode and/or lead and may increase the efficiency of the device, such as a cardiac pacemaker. Furthermore, increasing the efficiency of the device, such as a cardiac pacemaker, may lead to decreased power consumption, longer battery life, fewer procedures, better biocompatibility, and lower costs. The surface coating may be conductive, nonfibrotic, biologically inert, drug-free, or a combination thereof. In embodiments, the coated electrode may reduce the voltage, amperage, frequency of signal, or some combination thereof, needed to satisfactorily stimulate the tissue, as compared to uncoated electrodes of otherwise similar design. For example, in cardiac applications, the coated electrode may reduce the pacing threshold (PT) signal required to stimulate the heartbeat to the actual required level, or closer to the actual required level, as compared to prior art electrodes, which require the use of a PT that is two to three times greater than what is actually necessary.

In some embodiments, electrodes and/or leads may be used in conjunction with cardiac pacemakers; neural stimulation devices, such as deep brain stimulation devices that may be used to treat depression, Parkinson's Disease, insomnia, and the like; cosmetic electrotherapy devices; neuromuscular electrical stimulation devices that may be used in the treatment of neuromuscular disorders, dysphagia, and the like; and other indications requiring or benefiting from electrical stimulation or electrical connection to bodily tissues.

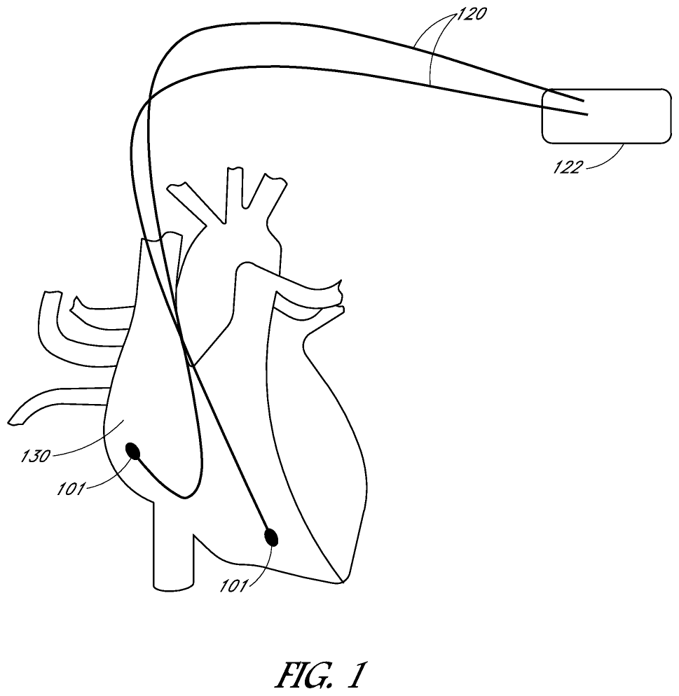

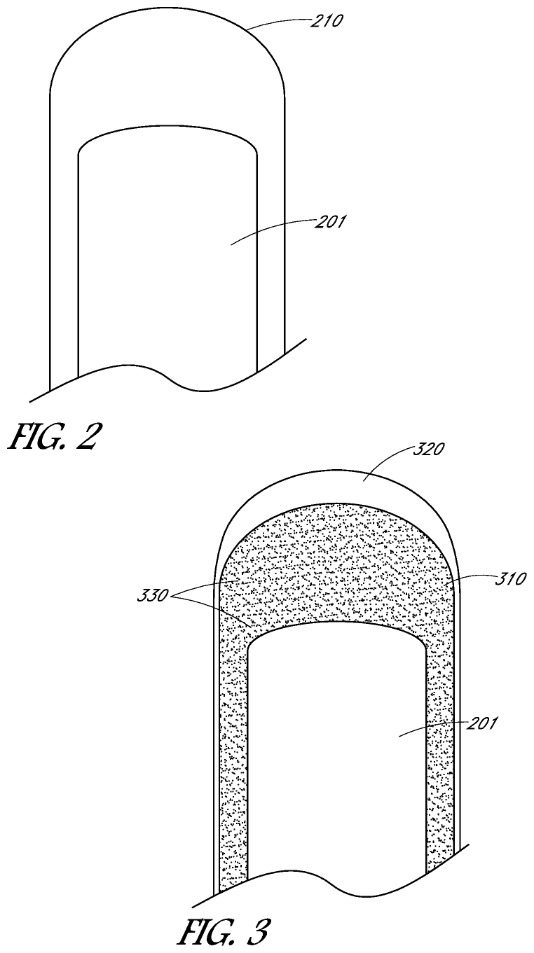

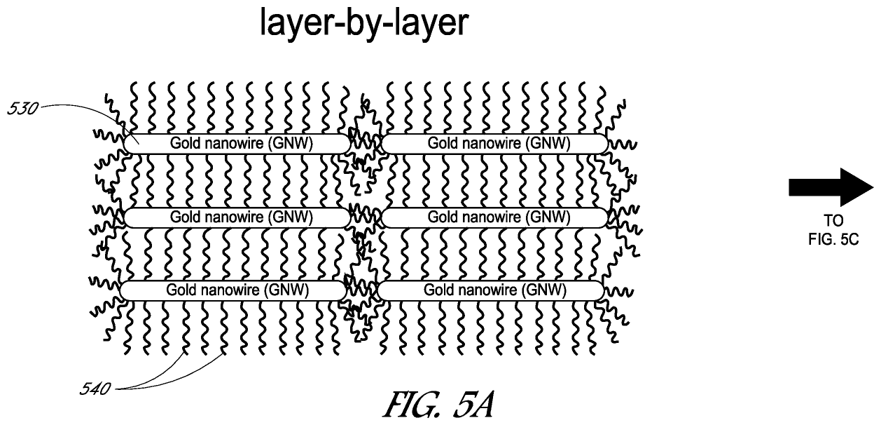

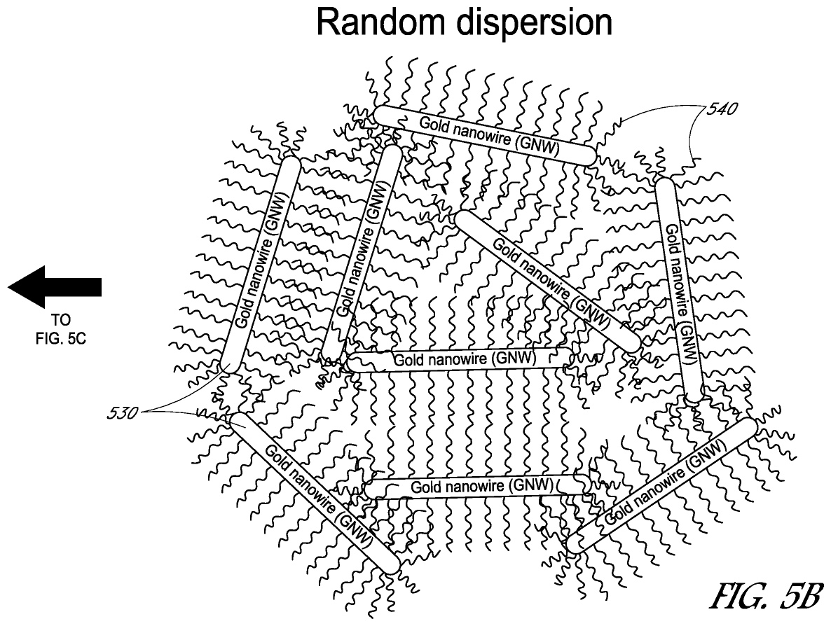

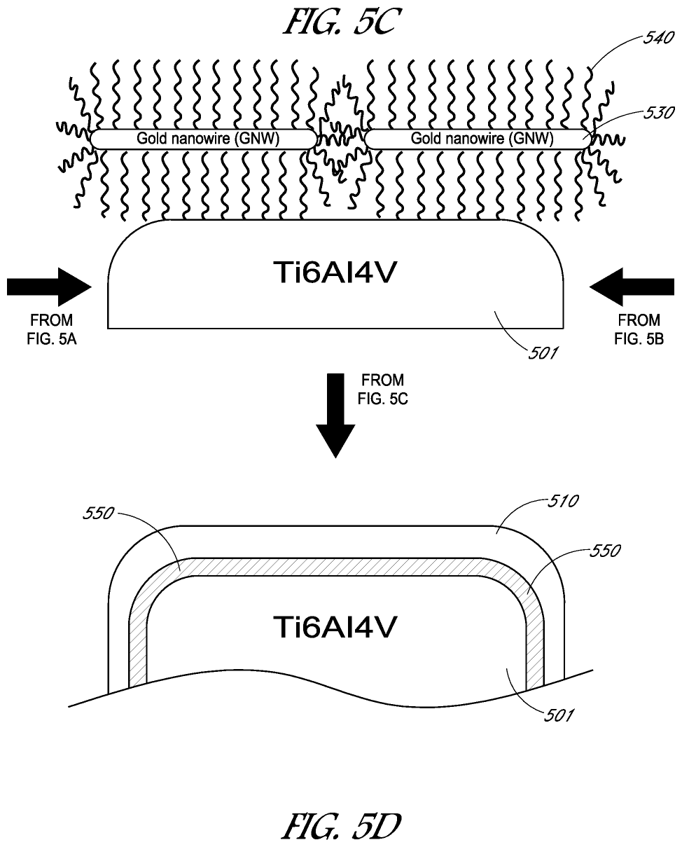

In some embodiments, a non-fibrotic, conductive, implantable cardiac pacemaker electrode lead (see, e.g., 120 in FIG. 1), neural stimulation lead, or muscular stimulation lead, comprising an electrode (see, e.g., 101 in FIG. 1, and 201 in FIGS. 2-4), such as a Ti-6Al-4V electrode (see, e.g., 501 in FIGS. 5c and d), an electrically conductive material, such as gold, and a non-fibrotic material, such as poly(ethylene) glycol, is described herein. The components of the novel electrode and/or lead coating may biologically compatible. The novel coated electrode and/or lead may have the following characteristics: a) the electrode may be capable of successfully delivering an electrical current to support contractility of cardiomyocytes, successfully delivering a therapeutic electrical current to neural tissues, or successfully delivering a therapeutic electrical current to muscle tissues; b) the presence of electrically conductive material embedded or dispersed in non-fibrotic material may conduct the electrical signal from the electrode to the surface to cardiomyocytes; and c) the inert coating of the non-fibrotic material may prevent, lessen, or ameliorate a fibrotic response from tissue surrounding the electrode and/or lead. The coated electrode and/or lead may exhibit these characteristics without the need for drugs, of with reduced size or number of doses of drugs as compared to those commonly used with uncoated electrodes and/or leads of otherwise similar design.

In some embodiments, the electrode with coating may deliver a therapeutic electrical current without inducing a fibrotic response in the surrounding tissue or inducing a lesser fibrotic response compared to an uncoated electrode of otherwise similar construction. The electrode with coating may deliver electrical impulses to tissue with the same intensity and/or efficiency as an uncoated electrode of otherwise similar construction or may deliver electrical impulses with the less intensity and/or efficiency as an uncoated electrode of otherwise similar construction, but still deliver electricity sufficiently to perform its intended function. Moreover, the characteristics of the current delivered to the electrode may be adjusted to compensate for any reduction in efficiency and/or intensity imparted by the coating. For example, voltage, amperage, current, and/or frequency of stimulation, values may be adjusted. A lead with coating may carry a therapeutic electrical current without inducing a fibrotic response in the surrounding tissue or inducing a lesser fibrotic response compared to an uncoated lead of otherwise similar construction.

In some embodiments, the coated electrode and/or lead may deliver and/or conduct a therapeutic electrical current without inducing a fibrotic response in the surrounding tissue or inducing a lesser fibrotic response, compared to an uncoated electrode and/or lead of otherwise similar construction, without the use of steroids, such as steroid elutions, or other drugs. However, in certain embodiments, the coated electrode and/or lead may be used in conjunction with steroids, such as steroid elutions, or other drugs. In certain embodiments, the dose of drug needed to prevent or reduce fibrosis with the coated electrode and/or lead may be less than the dose needed to prevent or reduce fibrosis with an uncoated electrode and/or lead of otherwise similar construction.

In some embodiments, a biologically stable implant electrode and/or lead is provided. In some embodiments, the electrode may be one that is capable of successfully delivering an electrical current to support contractility of cardiomyocytes, capable of successfully delivering a therapeutic electrical current to neural cells, and/or capable of successfully delivering a therapeutic electrical current muscle cells. In some embodiments, the electrode may comprise materials that are biocompatible and/or exhibit reduced immunogenicity and/or reduced fibrotic tendencies. “Biocompatible material(s)” as used herein may refer to materials that do not cause physical trauma or that cause minimal physical trauma; materials that are non-toxic or of low toxicity; materials that are not physiologically reactive or are minimally physiologically reactive; and/or materials that are not immunologically reactive or are minimally immunologically reactive. “Biocompatible material(s)” as used herein may refer to materials that are or can be approved by the Food and Drug Administration (“FDA”). In some embodiments, the electrode may be a TI-6AL-4V electrode or other electrode comprising titanium, or an Ir(IV)O₂ electrode or other electrode comprising iridium. In some embodiments, the electrode may comprise one or more of titanium, platinum, silicon, iridium, or carbon. Any electrode suitable for the selected application may be used in embodiments. An electrode may comprise an anode and/or a cathode. Any electrode lead suitable for the selected application may be used in embodiments. An electrode lead may comprise internal conductive wires covered by, coated by, or embedded in an insulative material, which may be biocompatible. Examples of electrodes and/or leads include electrodes with a standard terminal end, unipolar electrodes (as may be used, for example, in a unipolar pacing system), dipolar electrodes (as may be used, for example, in a dipolar pacing system), and quadripolar leads that contain four ring-shaped electrodes.

In some embodiments, the electrode tip or other portions of the electrode may be coated with a coating as described herein; i.e., a coating comprising a non-fibrotic material and/or an electrically conductive material. The coating may cover all, most, or a portion of the electrode that comes into contact with tissue after implantation. The entirety or portions of the electrode lead, including those portions that may come into contact with tissue after implantation, may be coated with a non-fibrotic material coating as described herein, or with a non-fibrotic material-electrically conductive material coating as described herein, or with another biocompatible material.

The coating may comprise a component that prevents or reduces adhesion and/or adsorption of proteins on its surface, which may prevent fibrosis of or in tissue surrounding the electrode and/or lead after it is implanted or placed in contact with tissue. This component may be referred to herein as a “non-fibrotic material”. The non-fibrotic material may comprise a biologically inert material. The non-fibrotic material may comprise a biocompatible material. The non-fibrotic material may comprise a hydrogel that lacks cell adhesion moieties. The non-fibrotic material may comprise poly(ethylene) glycol (PEG). The non-fibrotic material may comprise poly(ethylene) glycol, or modified poly(ethylene) glycol, such as thiolated poly(ethylene) glycol. Many suitable molecular weight PEGS may be utilized. For example, PEG having molecular weight of 1,000-20,000 may be utilized. For example, PEG3400 may be utilized. The molecular weight PEG that is utilized may be optimized to optimize characteristics such as the stiffness and porosity of the hydrogel. In some embodiments, the non-fibrotic material or hydrogel that lacks cell adhesion moieties may not comprise alginate.

In some embodiments, the coating may comprise an electrically conductive material. The electrically conductive material may be tunable, such as by adjusting its size (such as length and/or diameter), concentration, and/or organization. The electrically conductive material may comprise nanowires (which may be approximately 1-2 micron in length), nanorods (which may be approximately 100 nm in length), nanoparticles or nanospheres (which may be approximately 50 nm in diameter or length), or sizes in between. Wires, nanowires, rods, nanorods, particles, and nanoparticles may all be referred to as “particles” herein. The electrically conductive material may preferably comprise gold. The electrically conductive material may comprise gold nanowires (GNW), gold nanorods (GNR), or gold nanoparticles (GNP). The electrically conductive material may also comprise carbon or graphene oxide.

In some embodiments, the molecular weight and/or concentration of the non-fibrotic material; the concentration of the electrically conductive material; the size (such as length and/or diameter) of particles of the electrically conductive material; and/or the organization (such random dispersions, organized dispersions, or layer-by-layer constructions) of the electrically conductive material may be optimized to tune the mechanical properties, the porosity, the non-fibrotic tendencies, and/or the electrical conductivity of the coating. As an example, increasing the concentration of GNWs (such as from 0.5 mg/ml to 1.5 mg/ml) may enhance the contractility of cardiac cells.

In embodiments, the coating may comprise electrically conductive material dispersed in non-fibrotic material. Such a dispersion may be referred to herein as a “dispersion” or as a “mixture”. The dispersion may be random or ordered. The dispersion may form a structure, such as a lattice. An exemplary random dispersion, using PEG and GNW, is depicted in FIG. 5B.

In embodiments, the coating may comprise an inner layer of electrically conductive material dispersed in non-fibrotic material, and this dispersion layer may be coated or partially coated with an outer layer of non-fibrotic material that does not comprise electrically conductive material. In so doing, the layer of non-fibrotic material that does not comprise electrically conductive material may be outside of the layer of dispersion, relative to the electrode. The layer of non-fibrotic material that does not comprise electrically conductive material may be thinner or thicker than the layer of dispersion. In certain embodiments, it may be preferred that the layer of non-fibrotic material that does not comprise electrically conductive material be thinner than the layer of dispersion. Optionally, an innermost layer of layer of non-fibrotic material that does not comprise electrically conductive material and that is bound to an electrode via a metal binding protein, such as a titanium binding protein, or other suitable surface chemistry, may be included. However, optionally, the inner layer of dispersion may be bound directly to the electrode via suitable surface chemistry.

Layers may have many suitable dimensions, such as thicknesses. For example, thicknesses may be 100-200 microns, up to approximately a few hundred microns, less than approximately 500 microns, less than approximately 1 mm, or less than approximately 2 mm, or less than approximately 3 mm. For example, a thinner layer may be less than approximately a few hundred microns in thickness, less than approximately 500 microns in thickness, less than approximately 1 mm in thickness, or less than approximately 1.5 mm in thickness, whereas a thicker layer may be approximately 1.5-2 times the thickness of the thinner layer.

In embodiments, dispersions may contain various amounts, densities, or concentrations of electrically conductive material dispersed in the non-fibrotic material. For example, electrically conductive material may be dispersed in non-fibrotic material at concentrations of approximately 0.5 mg/ml to 2 mg/ml. The coating may comprise an inner layer of dispersion having a first concentration of electrically conductive material, and this inner dispersion layer may be coated or partially coated with an outer layer of dispersion comprising a second concentration of electrically conductive material. The first and second concentrations may be different. The first concentration may be greater or lesser than the second concentration. In certain embodiments, it may be preferred that the first concentration is greater than the second concentration. The inner and outer layers may have different thicknesses. Optionally, an innermost layer of layer of non-fibrotic material that does not comprise electrically conductive material and that is bound to an electrode via a metal binding protein, such as a titanium binding protein, or other suitable surface chemistry, may be included. However, optionally, the inner layer of dispersion may be bound directly to the electrode via suitable surface chemistry.

In embodiments, different numbers of layers having different compositions and/or thicknesses may be employed in a coating, such as three layers, four layers, or more layers. In embodiments, the coating may comprise a layer-by-layer (LBL) construction, which may comprise one or more layers of non-fibrotic material alternating with one or more layers of electrically conductive material. In such a layer-by-layer construction, the layers may comprise very thin layers, such as a layer of single molecules of non-fibrotic material, with a layer of single particles of electrically conductive material thereon, and a layer of single molecules of non-fibrotic material thereon, and so on. An exemplary layer-by-layer construction, using PEG and GNW, is depicted in FIG. 5A.

In embodiments, layer-by-layer constructions may be combined with layers of dispersions and/or layers of non-fibrotic material, in various combinations. In embodiments, an innermost layer may comprise non-fibrotic material or electrically conductive material conjugated to the electrode surface. Other layers, such as (i) layer(s) of dispersions of electrically conductive materials in non-fibrotic materials, (ii) layer(s) of non-fibrotic materials, and/or (iii) layer(s) of electrically conductive materials, may be layered thereon, in various combinations. In one embodiment, an innermost layer may comprise non-fibrotic material conjugated to the electrode surface, a second inner layer of electrically conductive material dispersed in non-fibrotic material or electrically conductive material only may be layered thereon, and a third outer layer of non-fibrotic material may be layered thereon. In embodiments, it may be preferred that the outermost layer of material of the coating, relative to the electrode, may be layer of non-fibrotic material. This outermost layer may be thin or very thin, such as approximately one to ten molecules thick. The coating may be conjugated or otherwise bound to the electrode via any suitable surface chemistry or linker. The method of conjugation or binding may be chosen based on its ability to adhere the coating to the electrode and/or remain stable under physiological conditions after implantation. For example, a peptide that binds the material of the electrode and also binds the coating may be employed. For example, a metal binding peptide, such as a titanium binding peptide, may be employed.

Referring to FIG. 1, electrode lead(s) 120 may be electrically coupled with a pacemaker 122, and the electrode lead(s) 120 may be implanted within a heart 130. Electrode leads 120 may end in or otherwise comprise electrode(s) 101. In an unshown embodiment, electrode lead(s) 120 and electrode(s) 101 may be implanted into a brain or other neural structure, into muscle, or into still other tissue types. Referring to FIG. 2, an electrode 201 may have a novel coating 210 coated thereon. The novel electrode material and coating may retain the necessary conductive functionality of a pacemaker or neurostimulator electrode but may inhibit a fibrotic response without the use of drugs, such as steroid elution. Coating 210 may comprise, for example, a non-fibrotic material. Coating 210 may comprise, for example, a dispersion of electrically conductive material dispersed in a non-fibrotic material. Coating 210 may comprise, for example, layers of non-fibrotic material alternating with layers of electrically conductive material. In such embodiments, the outer layer may be non-fibrotic material.

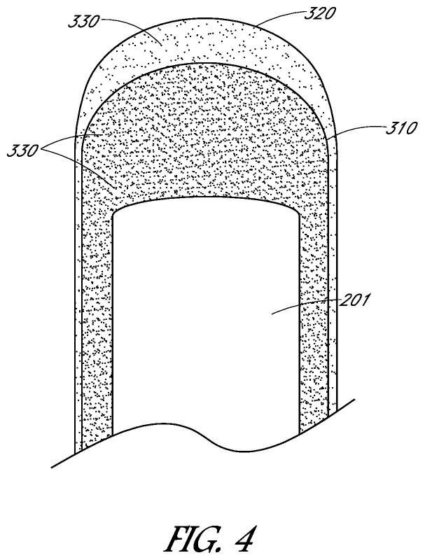

Referring to FIG. 3, the coating on electrode 201 may have two or more layers, which may be of different compositions and different thicknesses. For example, inner layer 310 may comprise a dispersion of electrically conductive material 330 dispersed in a non-fibrotic material, and inner layer 310 may be thicker than outer layer 320, which may comprise a non-fibrotic material. Further alternative embodiments are possible.

As a further example, referring to FIG. 4, inner layer 310 may comprise a dispersion of electrically conductive material 330 dispersed in a non-fibrotic material, and inner layer 310 may be thicker than outer layer 420, which may comprise a dispersion of electrically conductive material 330 dispersed in a non-fibrotic material, wherein the concentration of electrically conductive material 330 in outer layer 420 may be lesser than the concentration of electrically conductive material 330 in inner layer 310. In any layer comprising electrically conductive material, although a random dispersion is shown in the Figures, an ordered or structured dispersion may be employed.

Although not shown, in FIGS. 2, 3 and 4, coating 210 or inner layer 310 may be bound, conjugated, or otherwise immobilized to electrode 201 via a peptide that binds both the electrode and the coating, or by another suitable surface chemistry. In any of FIG. 2, 3, or 4, optionally, an unshown innermost layer of layer of non-fibrotic material that does not comprise electrically conductive material and that is bound to electrode 201 via suitable surface chemistry, such as a metal binding protein, such as a titanium binding protein, may be included. However, coating 210 or inner layer 310 may be bound directly to electrode 201 via suitable surface chemistry, such as a metal binding protein, such as a titanium binding protein. Still more layers of coating, having various compositions, organizations, and thicknesses, may be employed, in embodiments. For example, although dispersions are depicted in FIGS. 3 and 4, layer-by-layer constructions may be used in coatings, including in combination with dispersion layers.

FIG. 5a illustrates development of a GNW-PEG coating with a layer-by-layer construction using poly(ethylene) glycol (PEG) 540 layered with gold nanowires (GNW) 530, in accordance with some embodiments. FIG. 5b illustrates development of a GNW-PEG coating with a random dispersion of GNW 530 in PEG 540 matrix, in accordance with some embodiments. FIG. 5c illustrates development of a titanium electrode 501 coated with a layer-by-layer construction of PEG 540 layered with GNW 530, in accordance with some embodiments. Although not shown, the PEG may be conjugated or bound to the titanium electrode using any suitable surface chemistry, such as a titanium binding peptide (TBP) that also binds PEG.

FIG. 5d illustrates development of a titanium electrode 501 coated with a coating 510, in accordance with some embodiments. The coating may comprise a layer-by-layer and/or random dispersion construction. The coating may comprise more than one layer. The coating may be conjugated or bound to the titanium electrode using any suitable surface chemistry 550. Surface chemistry 550 may comprise metal binding proteins (MBP), such as titanium binding peptides (TBP), that also bind the coating 510. The MBP or TBP may bind the PEG in the coating 510. The coating and/or layer(s) thereof may take various shapes. In embodiments, the coating layer(s) conform to the shape of the electrode and/or lead. Each coating layer may have a uniform thickness or may have varying thicknesses. As explained earlier herein, preferably at least the portion of the electrode that will be in contact with tissue after implantation is coated. The portion of the lead that will be in contact with tissue after implantation may be coated. In embodiments, elimination or reduction of fibrotic response may be enhanced through use of drugs, such as steroid elution systems or chambers near the electrode.

In embodiments, electrodes may include optimizations in addition to the coatings described herein. Electrodes may have optimized surface texture. Electrodes may have a porous surface or may have a relatively non-porous surface. Electrodes may have varying surface areas. Electrodes may have optimized dimensions, such as length, diameter, Electrodes may have optimized shapes, such as cylindrical, rod-shaped, spherical, pad-shaped, tined, or hooked. Electrodes may comprise surface treatments in addition to the coatings described herein, such as alginates, endogenous protein ligands, covalent grafting, and glow discharge plasma disposition treatments. Such treatments are not considered to be coatings as described herein and are not considered to be coatings for purposes of comparing the performance of coated electrodes to electrodes of similar construction but lacking coating. In embodiments, the properties of electrodes may be adjusted, either before or after coating, to assist in eliminating or reducing the fibroblast response. In some embodiments, the electrode may comprise titanium, such as a Ti-6Al-4V electrode, the non-fibrotic material may comprise PEG, and the electrically conductive material may comprise gold.

In some embodiments, gold may be modified and may readily self-assemble to thiolated compounds without requiring complex steps. For example, GNWs may be PEGylated, where one side is the thiol group (—S) which attaches to the gold and the other side can be attached to other ligands such as carboxylic groups (—COOH). Anchoring a thiol group to a desired ligand may impart a stealth character to GNR, GNP, or GNW and may replace the cytotoxic hexadecyltrimethylammonium bromide (CTAB) coating that is affixed to the GNR, GNP, or GNW during seed-mediated synthesis. A variety of thiol-PEG structures may readily assemble on the surface of various sizes and shapes of GNP, GNR, or GNW. A variety of molecular weight PEG bound to different sizes of GNP have demonstrated biocompatibility. Generally, PEGylated GNP, GNR, or GNW with molecular weight (MW) greater than approximately 1000 Daltons exhibit a neutral charge at the surface.

Proteins that adsorb or adhere onto the surface of electrodes may facilitate fibrosis of the tissue. PEGylation of GNRs, GNWs, and/or GNPs may render them extremely stable and resistant to human skin and proteins These PEGylated GNP, PEGylated GNW, or PEGylated GNR particles, with an aspect ratio (AR) of approximately (˜) 4-75, may exhibit steric hindrance at their surface that may render them neutral. Without being bound by theory, it is believed that this property occurs because PEGylation forms dense, brush-like structures that resist protein adsorption and protein adhesion. PEG has also been investigated for its anti-thrombotic characteristic as a surface coating and modifying the free end of the PEG chain with a methyl group may render it inert. Thus, the synthesis of a PEG-GNR, Peg-GNW, or PEG-GNP nonfibrotic, conductive surface coating may be an excellent candidate material for the next generation of electrode materials.

In some embodiments, surface coatings may be synthesized with components comprising gold nanowires (GNW) and poly(ethylene) glycol (PEG) using a layer by layer and/or a random dispersion design, as illustrated in FIGS. 5a-5d.

GNR-PEG, GNP-PEG, and GNW-PEG surface coatings may be produced by any suitable method. In some embodiments, GNW may be formed from seed-mediated synthesis and reduction of HAuCl₄.

The following steps may be performed to develop the layer-by-layer and/or random dispersion GNW-PEG surface coating: (a) Synthesis of GNP seeds; (b) Synthesis of GNW; (c) Synthesis of layer-by-layer GNW-PEG, or (d) Synthesis of random dispersion GNW-PEG.

(a) Primary synthesis of GNP seeds: GNP seeds may be produced by any suitable method. GNW may be formed from seed-mediated synthesis and reduction of HAuCl₄. One non-limiting method of GNP seed synthesis is briefly described as follows: Dissolve a 0.2 M solution of hexadecyltrimethylammonium bromide (CTAB) in deionized water (DIW). Add 2 mL of 1 mM solution of chloroauric acid (HAuCl₄) directly to 2 mL CTAB solution. Prepare an ice-cold 0.01 M solution of the reducing agent, sodium borohydride (NaBH₄). While vortexing the CTAB-HAuCl₄ mixture, add 240 μL of 0.01 Mat once and continue to shake for 1 minute. The particles will have dimensions of a few nanometers and the solution will turn brown with successful formation. Allow the solution to rest at RT for at least 30 minutes.

(b) Synthesis of GNW: GNW may be produced by any suitable method. One non-limiting method of GNW synthesis is briefly described as follows: A procedure to control the length and aspect ratio of GNW is done using the GNP previously synthesized. An HNO₃-mediated growth solution provides an anisotropic growth environment for the GNW to assemble: 29.475 mL of DIW, 1.093 g of CTAB, and 525 μL of 0.01 M HAuCl4. The growth solution is distributed into 3 flasks: 2.5 ml into both flasks A and B, and the remaining 25 ml into flask C. Add 5 mL of 0.5 M solution of nitric acid (HNO3) into flask C. Using a weaker reducing agent may allow the growth solution to incorporate with the GNP to grow into GNW. Volumes of 0.1 M Ascorbic Acid (AA) are added to flasks A, B, and C, at 10, 10, and 100 μL respectively. The flasks are gently shaken until they are clear. Add 200 μL of gold seed solution to flask A and shake for 10 sec. Transfer 200 μL from flask A into flask B and shake for 10 sec. Transfer 200 μL of solution from flask B into flask C and shake for 5 sec. Store the resulting solution, undisturbed, for a minimum of 12 hours for completion of GNW growth. Remove the upper solution that contains the GNP. Re-suspend the GNW at the bottom of the flask in 10 mL of DIW. Concentrate the GNW by two cycles of centrifugation at 200 rpm for 20 min.

(c) Synthesis of layer-by-layer GNW-PEG: Layer-by-layer GNW-PEG may be produced by any suitable method. One non-limiting method of layer-by-layer GNW-PEG synthesis is briefly described as follows: Thiolated pegylation can self-assemble on GNW and displace the toxic CTAB coating. PEG is immobilized on to a gold surface slide. Briefly, 1.0 mL of twice cleaned CTAB-GNW solution is combined with 0.1 mL of SH-PEG-SH solution (10 mg/mL) and mixed for 12 h. Excess polymer is removed by centrifugation 2× at 10,000 rpm for 10 min. This process is repeated in order to assemble 5 layers of GNW.

(d) Synthesis of random dispersion GNW-PEG: Random dispersion GNW-PEG may be produced by any suitable method. One non-limiting method of random dispersion GNW-PEG synthesis is briefly described as follows: The random dispersion technique may incorporate the GNW into a matrix of PEG. Studies may be conducted to determine whether there is a difference in the mechanical, conductive, or biocompatibility of random dispersion vs. layer-by-layer surface coating. Briefly, 1 mL of SH-PEG-SH solution is added to of 5 mL of GNW solution (˜1×10¹⁹ GNW/L). The reaction mixture is stirred for 24 h, dialyzed (MWCO membrane 5 kDa) for three days with four water changes per day against ultrapure water. It is then washed three times by centrifugation for 15 min at 10,000 rpm and concentrated.

The GNW-PEG, GNR-PEG, GNP-PEG surface coating, or other surface coating comprising non-fibrotic material and/or electrically conductive material, may comprise any or all of the following characteristics: mechanical stability, mechanical robustness, structural integrity, and other mechanical properties; conductivity and other electrical properties; porosity; and biocompatibility. Because both electrically conductive materials, such as GNW, and non-fibrotic materials, such as PEG, are highly tunable for size, molecular weight, and functionality, and various permutations and combinations (such as changes in thickness, concentration, and organization) may be made, coatings may be optimized for various uses.

In some embodiments, the developed coatings may be characterized in terms of mechanical stability, mechanical robustness, structural integrity, and other mechanical properties; conductivity and other electrical properties; porosity; and biocompatibility. The electrical, structural, and mechanical properties of coatings may be characterized prior to anchoring to an electrode surface. Likewise, coated electrodes may be characterized in terms of mechanical stability, mechanical robustness, structural integrity, and other mechanical properties; conductivity and other electrical properties; porosity; and biocompatibility. The electrical, structural, and mechanical properties of coated electrodes may be characterized prior to use. Coated leads may likewise be optimized and tested.

GNW micrographs may be obtained using transmission electron microscopy (TEM) (Philips CM200-FEG, USA) operating at an accelerating voltage of 200 kV. The energy dispersive X-ray spectroscopy (TEM-EDX, Philips CM200-FEG, USA) may be used to further confirm the presence of GNW within the PEG matrix. UV-Vis absorption spectra may show longitudinal and transverse surface plasmon (SPR) peaks. The microstructure of the constructs may be evaluated by means of scanning electron microscopy (SEM) (XL30 ESEM-FEG, USA). The Young's modulus value for mechanical stiffness may be evaluated by atomic force microscopy (AFM) (MFP-3D AFM, Asylum Research) with silicon nitride tips (MSNL, Bruker). For impedance analysis, the constructs may be located between two indium tin oxide (ITO) coated glass slides (Sigma-Aldrich) with an AC bias sweeping (Agilent 4284A LCR meter) from 20 Hz to 1 MHz.

The coatings, such as GNW-PEG, may be biocompatible and conductive. The conductivity of the surface coating may be varied by varying the concentration and/or number of layers of electrically conductive materials, such as GNW, in non-fibrotic materials, such as PEG. For example, the conductivity may be increased by increasing the concentration and/or number of layers of GNW in PEG. The conductivity may be adjusted to produce a satisfactory PT. The self-assembling tendency of GNW to thiolated PEG will may allow a broad range of tunability options for the coating material. Additionally, a mixed thiol monolayer (e.g., thiolated PEG and cystamine) may also be used to displace CTAB. The cystamine may also be added after pegylation. PEG-Cys-GNW has been shown to exhibit a higher zeta potential than PEG-GNW.

In some embodiments, the surface coating may be applied to an electrode, and the characteristics and biocompatibility of the completed electrode construct on cardiomyocytes and fibroblasts may be assessed.

In some embodiments, a titanium-alloy (e.g., Ti-6Al-4V) material may be used for both bone implantation as well as the surface coating for neural conductive electrodes and/or leads. A polyethylene glycol (PEG) construct with modified chemistry may be tethered to the titanium by a surface binding peptide to the titanium. This technique may be used to prevent fouling in implantable devices by preventing cellular adhesion. Gold nanorods (GNR) can remain conductive but may induce a fibrotic or toxic response unless coated with or dispersed in an inert, biocompatible material.

In some embodiments, the electrically conductive material non-fibrotic material (such as GNW-PEG) constructs may be conjugated to an electrode that is capable of successfully delivering an electrical current to support contractility of cardiomyocytes. The electrode may be a Ti-6Al-4V electrode.

In some embodiments, Ti-6Al-4V may be utilized as a long-term, non-corrosive orthopedic device that has conductive character. Ti-6Al-4V can also effectively bind biocompatible PEG via a TBP, and multivalent binding of PEG to GNW may increase the overall molecular stability.

By embedding electrically active material, such as GNW, into non-fibrotic material, such as PEG, the conductivity of the electrode, such as a Ti-6Al-4V electrode, may be retained through the non-fibrotic material, while also preventing the electrode from creating a biological response. Attaching functional groups to non-fibrotic material, such as PEG, may allow it to be attached to the surface of an electrode, such as a Ti-6Al-4V electrode, surface, and may leave the free end available to add on the electrically active material-non-fibrotic material constructs, such as GNW-PEG constructs, synthesized.

The following steps are an exemplary method that may be used to a develop Ti-GNW-PEG nonfibrotic, conductive electrode: (a) Immobilize TBP-PEG-(SH) onto Ti-6Al-4V surface; (b) attach previously synthesized GNW-PEG constructs to Ti-bound thiol terminated TBP-PEG.

(a) Immobilize PEG onto Ti-6Al-4V surface: PEG may be immobilized by any suitable method. One non-limiting method of immobilization of PEG is via a tetravalent titanium-binding peptide (TBP) such as SHKHGGHKHGGHKHGSSGK (SEQ ID NO: 1). This TBP binds to PEG, forming SHKHGGHKHGGHKHGSSGK-PEG-(SH), and binds to titanium, as well, forming a PEG-coated titanium surface. Other metal binding proteins are known, including those set forth in U.S. Pat. No. 7,972,615, which is incorporated herein in its entirety.

(b) Electrically active material-PEG constructs, such as GNW-PEG constructs, will then be attached to Ti-bound thiol terminated TBP-PEG, as previously described, or by another suitable method.

Electrically active material-PEG-Ti constructs, such as Ti-GNW-PEG structures, may be synthesized easily and robustly with this method.

In this embodiment, a layer of PEG is immobilized onto a titanium surface, such as a Ti-6Al-4V surface, then Ti-GNW-PEG is layered thereon. However, Ti-GNW-PEG dispersion, or other dispersion of electrically conductive material in non-fibrotic material may be conjugated to directly to electrodes, such as titanium-based electrodes, using peptides or other surface chemistry that bind both (i) the non-fibrotic material and/or the electrically conductive material and (ii) the electrode surface.

In some embodiments, the properties of the developed Ti-GNW-PEG may be characterized in terms of electrical conductivity, structural integrity, and mechanical robustness may be characterized. In some embodiments, upon synthesis, the electrical, structural, and mechanical properties of the coated electrode surface, such as a Ti-GNW-PEG electrode surface, may be characterized. The electrode surface may be nonfibrotic and/or conductive. Properties may be compared to previous findings to confirm the finished surface coating conforms to desired criteria. The same methods previously used to determine the characteristics of the coating, such as GNW-PEG, alone may be employed in order to compare data consistently across the biomaterials.

GNW micrographs may be obtained using transmission electron microscopy (TEM) (Philips CM200-FEG, USA) operating at an accelerating voltage of 200 kV. Energy dispersive X-ray spectroscopy (TEM-EDX, Philips CM200-FEG, USA) may be used to further confirm the presence of GNW within the PEG matrix. UV-Vis absorption spectra may show longitudinal and transverse surface plasmon (SPR) peaks.

The microstructure of the constructs may be evaluated by means of scanning electron microscopy (SEM) (XL30 ESEM-FEG, USA). The Young's modulus value for mechanical stiffness may be evaluated by atomic force microscopy (AFM) (MFP-3D AFM, Asylum Research) with silicon nitride tips (MSNL, Bruker). For impedance analysis, the constructs may be located between two indium tin oxide (ITO) coated glass slides (Sigma-Aldrich) with an AC bias sweeping (Agilent 4284A LCR meter) from 20 Hz to 1 MHz.

In embodiments, the coated electrode, such as a Ti-GNW-PEG coated electrode, may be biocompatible, nonfibrotic, conductive, or any combination thereof. In order to develop more or most satisfactory coatings, synthesizing structures with varying concentration ratios of GNW (or other electrically conductive material) and PEG (or other non-fibrotic material) may be performed. Layer-by-layer coating may also be created through sequential electrostatic deposition of polylysine (PLL) and poly(L-glutamic acid) (PGA) grafted PEG (PGA-g-PEG). Coatings may also be further modified with anti-fibrotic substituents.

In some embodiments, the in vitro survival, retention, and spontaneous contractility of cardiac cells, or stimulation of other cells, in response to contact and stimulation by the coated electrode may be studied. Reducing the ECM's sensitivity to the electrode coating is important to eliminating the fibrotic response. The electrode may be mechanically and biologically stable, electrically conductive, resistant to biological adhesion, or any combination thereof. Upon synthesis of coated electrode materials, such as Ti-GNW-PEG biomaterials, they may be characterized and optimized. For example, cardiac, muscle, and neural tissues similar to native tissues may be microengineered in order to assess biocompatibility of the novel surface coating. The best layer-by-layer (LBL) or random dispersion surface coating candidates may be selected based on passing stability and conductivity criteria. The selected coatings may be used to perform extensive in vitro testing on tissue samples and in vivo testing in animal models. The performance of the selected electrode coatings may be evaluated based on the tissue's and animals' response.

For example, ventricular cardiomyocytes from neonatal rats may be cultured in order to assess biocompatibility and conductivity of the selected coated electrode material, such as Ti-GNW-PEG. Prior to seeding, the selected coated electrode material will be washed 2 times, 10 min intervals, in 1% (v/v) penicillin-streptomycin (Gibco, USA) in DPBS and then washed 2 times, 10 min intervals, in cardiac culture medium (DMEM) (Gibco, USA), 10% fetal bovine serum (FBS) (Gibco, USA), 1% L-Glutamine (Gibco, USA) and penicillin-streptomycin (100 U/mL). Harvested cardiomyocytes may be seeded on top of the selected coated electrode material constructs and cultured in cardiac specific media for 7 days under static condition.

Cardiomyocyte viability may be determined with standard Live/Dead assay kit (Life Technologies, USA) according to manufacturer's protocol. A fluorescent microscope (Zeiss Observer Z1) may be used to image the cells. Quantifications may be performed utilizing NIG ImageJ software. Metabolic activity of the cells on the constructs may be determined with the Alamar Blue assay kit (Invitrogen, USA) according to manufacturer's protocol. A fabricated chamber may be used to apply external electrical stimulations and the beating frequency (BPM) will be measured across a range of applied voltages. Immunocytochemistry may be used to visualize expressed fibrosis markers: collagen 1a1 (Col1a1), collagan 1a2 (Col1a2) and SMA.

Cells may be fixed and treated to permeabilize the plasma membrane. Once blocked, primary antibodies may be added. After three washing steps, the secondary antibodies (Life Technologies, USA) with protein specific dyes may be added. DAPI staining (1:1000 dilution in DPBS) may be used to label the nucleus. Images may either be viewed with a confocal microscope (Leica TCS SP5 APBS Spectral Confocal System) or a fluorescent microscope. Expression of fibrosis markers can also be evaluated through qPCR.

In some embodiments, the geometrical features (i.e. length, thickness, diameter) as well as the concentration and/or organization of the electrically conductive material, such as GNRs, conjugated within the non-fibrotic material, such as PEG hydrogel, may be optimized. Specifically, increasing size and/or concentration of the electrically conductive material, such as increasing the length of the GNRs, from nanorods toward nanowires, may increase conduction within the hydrogel layer in between the electrode and the contact point with the beating cardiomyocytes or other tissue to be stimulated. Increasing the size and/or concentration of the electrically conductive material may induce more nanoscale surface topographies within the hydrogel matrix in favor of cardiac fibroblasts or other tissue attachment and spreading which may lead to fibrosis formation around the electrodes and/or leads. There may be a balance between the size and/concentration and geometries of the electrically conductive material to induce sufficient conductivity while sufficiently inhibiting tissue fibrosis. The properties of the non-fibrotic material, e.g., PEG, such as its concentration and/or molecular weight, may also be optimized.

Different thicknesses of coating layers, numbers of coating layers, and compositions of coating layers on electrodes may also be investigated. For example, a thicker inner layer of non-fibrotic material electrically conductive material having a first concentration of electrically conductive material dispersed therein, in turn coated with an outer thinner layer of non-fibrotic material having no, or a lesser concentration, of electrically conductive material dispersed therein may be found to provide a superior combination of properties, such as sufficient conductivity of electrical impulses to tissue combined with sufficient reduction or elimination in fibrosis of tissue in contact with the coating on the electrode. Optionally, an innermost layer of layer of non-fibrotic material that does not comprise electrically conductive material and that is bound to an electrode via a metal binding protein, such as a titanium binding protein, or other suitable surface chemistry, may be included. However, optionally, the inner layer of dispersion may be bound directly to the electrode via suitable surface chemistry.

Moreover, the characteristics of the electricity delivered by the electrode may be adjusted to compensate for any reduction in efficiency and/or intensity imparted by the coating. For example, voltage, amperage, frequency of stimulation, direct voltage, alternating voltage, direct current alternating current, and/or other electrical variables may be adjusted. PT, for example, may be ±0.5V at 0.5 ms, or maybe more or less than this value.

Further, other peptides or other surface chemistries to ensure sufficient adhesion of the hydrogel coating to the metal of the electrodes may be used to address any lack of sufficient adhesion of the proposed coating to the metal surface (i.e., electrode) due to the lack binding of the proposed surface peptide).

While embodiments have been illustrated herein, it is not intended to restrict or limit the scope of the appended claims to such detail. In view of the teachings in this application, additional advantages and modifications will be readily apparent to and appreciated by those having ordinary skill in the art. Accordingly, changes may be made to the above embodiments without departing from the scope of the invention.

Various features, steps, processes, components, and subcomponents may be employed in certain embodiments. These features, steps, processes, components, subcomponents, partial steps, systems, devices, etc. may be adjusted, combined and modified in various fashions and various ways among and between the teachings and figures provided herein, as well as in other ways not specifically described herein but consistent with the teachings and discussion of this disclosure unless otherwise indicated herein or otherwise clearly contradicted by context. All methods described herein can be performed in any suitable order unless otherwise indicated herein or otherwise clearly contradicted by context.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the terms “about” or “approximately” in reference to a recited numeric value, including for example, whole numbers, fractions, and/or percentages, generally indicates that the recited numeric value encompasses a range of numerical values (e.g., +/−5% to 10% of the recited value) that one of ordinary skill in the art would consider equivalent to the recited value (e.g., performing substantially the same function, acting in substantially the same way, and/or having substantially the same result).

It should be noted that the terms “first”, “second”, and “third”, and the like may be used herein to modify elements performing similar and/or analogous functions. These modifiers do not imply a spatial, sequential, or hierarchical order to the modified elements unless specifically stated. Recitation of ranges of values herein are merely intended to serve as a shorthand method of referring individually to each separate value falling within the range, unless otherwise indicated herein, and each separate value is incorporated into the specification as if it were individually recited herein.

Certain embodiments may be implemented as a computer process, a computing system or as an article of manufacture such as a computer program product of computer readable media. The computer program product may be a computer storage medium readable by a computer system and encoding computer program instructions for executing a computer process.

The corresponding structures, material, acts, and equivalents of any means or steps plus function elements in the claims are intended to include any structure, material or act for performing the function in combination with other claimed elements. The description of certain embodiments of the present invention have been presented for purposes of illustration and description, but is not intended to be exhaustive or limited to the invention in the form disclosed. Many modifications and variations will be apparent to those of ordinary skill without departing from the scope and spirit of the invention. These embodiments were chosen and described in order to best explain the principles of the invention and the practical application, and to enable others of ordinary skill in the art to understand the invention for embodiments with various modifications as are suited to the particular use contemplated.

1. An implantable coated electrode where at least a portion of the electrode is coated with a coating, (i) the coating comprising a hydrogel lacking cell adhesion moieties, wherein the hydrogel comprises electrically conductive particles dispersed therein, or (ii) the coating comprising a hydrogel lacking cell adhesion moieties wherein the hydrogel has electrically conductive particles layered thereon.

2. The implantable coated electrode of claim 1, wherein the coating comprises two or more layers, at least a first of the two or more layers differing in composition, thickness, or both, as compared to at least a second of the two or more layers.

3. An implantable coated electrode at least a portion of which is coated with a coating, the coating comprising (1) an inner layer comprising a hydrogel lacking cell adhesion moieties, wherein the hydrogel comprises electrically conductive particles dispersed therein, and (2) an outer layer comprising a hydrogel lacking cell adhesion moieties, wherein the outer layer of hydrogel does not have electrically conductive particles dispersed therein.

4. The implantable coated electrode of claim 3, further comprising an innermost layer comprising a hydrogel lacking cell adhesion moieties.

5. The implantable coated electrode of claim 1, wherein the coating comprises a first layer of hydrogel lacking cell adhesion moieties, wherein the first layer of hydrogel has a first layer of electrically conductive particles layered thereon, wherein the first layer of electrically conductive particles has a second layer of hydrogel lacking cell adhesion moieties layered thereon.

6. The implantable coated electrode of claim 3, wherein the electrically conductive particles comprise gold.

7. The implantable coated electrode of claim 3, wherein the hydrogel lacking cell adhesion moieties comprises poly(ethylene) glycol.

8. The implantable coated electrode of claim 3, wherein the hydrogel lacking cell adhesion moieties comprises thiolated poly(ethylene) glycol.

9. The implantable coated electrode of claim 3, wherein the coating is immobilized on the electrode via a peptide that binds both the (i) electrode and (ii) the hydrogel, the electrically conductive material, or both.

10. The implantable coated electrode of claim 3, wherein the electrode comprises titanium, iridium, platinum, silicon, carbon, or a combination thereof.

11. A method of evaluating the performance characteristics of an implantable coated electrode of claim 3, wherein the performance characteristics comprise the electrical conductivity of the implantable coated electrode, the resistance to fibrosis of the implantable coated electrode, the biological stability of the implantable coated electrode, the mechanical stability of the coated electrode, or a combination thereof, the method comprising the steps of:
(a) (i) seeding cultured cells on one or more electrode coated with a first coating or
(ii) implanting one or more electrode coated with a first coating in an animal model, such that the one or more electrode is in contact with cells of the animal;
(b) electrically stimulating the cells seeded on the electrode or contacting the electrode in the animal model via the one or more electrode coated with the first coating; and
(c) observing one or more of the electrical conductivity, the resistance to fibrosis, the biological stability, and the mechanical stability of the one or more electrode coated with the first coating.

12. The method of claim 11, wherein the cells are observed for a period of approximately 4 weeks or the animal model is observed for a period of approximately 8-12 weeks.

13. The method of claim 11, wherein the cells are stimulated more than once.

14. The method of claim 11, further comprising the steps of:
(d) altering the one or more of the structure, number of layers, or composition of layers of coating to form a second coating;
(e) repeating steps (a)-(c) with one or more electrodes coated with the second coating; and
(f) comparing performance characteristics of the one or more electrode coated with the first coating to the performance characteristics of the one or more electrode coated with the second coating.

15. A method of preparing an implantable electrode coated with a coating comprising (1) a random dispersion of electrically conductive material in a hydrogel lacking cell adhesion moieties or (2) alternating layers of a hydrogel lacking cell adhesion moieties and electrically conductive material comprising the steps of:
(a) synthesizing of seeds of electrically conductive material;
(b) synthesizing nanowires of electrically conductive material;
(c) (i) dispersing the nanowires in the non-fibrotic material; and
(ii) immobilizing the non-fibrotic material comprising the nanowires dispersed therein on the surface of the electrode in one or more area(s) where the electrode, once implanted into a tissue, will be in contact with the tissue; or
(d) (i) immobilizing the non-fibrotic material or the nanowires on the surface of the electrode in one or more area(s) where the electrode, once implanted into a tissue, will be in contact with the tissue; and
(ii) layering the nanowires on the immobilized non-fibrotic material or layering the non-fibrotic material on the immobilized nanowires, alternating two or more layers of nanowires and non-fibrotic material.

16. The implantable coated electrode of claim 1, wherein the hydrogel lacking cell adhesion moieties comprises thiolated poly(ethylene) glycol.

17. The implantable coated electrode of claim 1, wherein the coating is immobilized on the electrode via a peptide that binds both the (i) electrode and (ii) the hydrogel.

18. The implantable coated electrode of claim 5, wherein the second layer of hydrogel lacking cell adhesion moieties has a second layer of electrically conductive particles layered thereon.


SEQUENCE LISTING (1 sequence)

SEQ ID NO: 1 - protein, 19 aa, Artificial
  Synthesized
SHKHGGHKHGGHKHGSSGK